(19) United States
(12) Patent Application Publication
(10) Pub. No.: US 2016/0278976 A1
(43) Pub. Date: Sep. 29, 2016

(54) THERMAL BAG FOR PROTECTING A NEWBORN

(71) Applicant: DRÄGERWERK AG & CO. KGAA, Lübeck (DE)

(72) Inventor: Thomas KRÜGER, Reinfeld (DE)

(21) Appl. No.: 15/032,807

(22) PCT Filed: Oct. 13, 2014
(86) PCT No.: PCT/EP2014/002753

§ 371 Date: Apr. 28, 2016

(30) Foreign Application Priority Data
Nov. 2, 2013 (DE) 10 2013 018 367.7

(51) Int. Cl. A61F 7/00; A41B 13/06; A61B 5/1455; A61B 5/00; A61B 5/0205; A61B 5/024

(52) U.S. Cl. CPC A61F 7/0097; A61B 5/02055; A61B 5/024; A61B 5/14552; A61B 5/0022; A41B 13/06; A61B 2503/045

(57) ABSTRACT

A thermal bag (10) for protecting a newborn (100) against hypothermia includes at least one insertion opening (20) for inserting the body (110) of the newborn (100) into an interior space (30) of the thermal bag (10). The interior space (30) is surrounded by a bag wall (40) blocking water vapor. The bag wall (40) has a sensor device (50) for determining at least one vital function of the newborn (100).

### CROSS REFERENCE TO RELATED APPLICATIONS

This application is a United States National Phase Application of International Application PCT/EP2014/002753 filed Oct. 13, 2014 and claims the benefit of priority under 35 U.S.C. §119 of German Patent Application 10 2013 018 367.7 filed Nov. 2, 2013 the entire contents of which are incorporated herein by reference.

### FIELD OF THE INVENTION

The present invention pertains to a thermal bag or thermoregulation bag for protecting a newborn against hypothermia as well as to a packaging unit for such a thermal bag.

### BACKGROUND OF THE INVENTION

It is known that there is a risk for hypothermia in newborns in different birth situations. This happens especially in case of premature births, in which the size of the newborn or the developmental status does not yet make possible a heat regulation of its own or does so to a limited extent only. It is also known that the newborn must be protected against intense hypothermia in such cases. A thermal bag, into which the newborn is inserted with his or her body up to the neck, is made available for this in the known manner. It can be ensured hereby that the hypothermia of the newborn's body, especially due to cooling by evaporation, is reduced or altogether avoided. Moisture, which evaporates from the skin of the newborn, saturates the relatively small volume of the thermal bag in the interior space of the latter, so that a further evaporation and the further hypothermia of the newborn's body, which is associated herewith, can be prevented.

The drawback of prior-art thermal bags is that these make additional treatments of the newborn difficult. In particular, it is often necessary to additionally monitor vital functions of the newborn's body, for example, the heart rate or the oxygen saturation in the blood. If, however, the newborn's body is within the thermal bag, the accessibility to the body is markedly restricted. This correspondingly leads to cumbersome solutions, i.e., to leading cables of corresponding sensor elements into the interior of the thermal bag. As an alternative, it is necessary to accept the absence of the detection of vital functions for the duration of the time the newborn is located within the thermal bag. This leads to increased risk, because the immediate care after delivery may possibly be carried out to a limited extent only without sufficient information on the corresponding vital function. It should be noted in this connection that the thermal bag represents an interim solution between the delivery room and the intensive care unit necessary for the newborn. For example, WO 2007/141490 A1 shows basically the possibility of using plastic bags as a thermal protection against cooling by evaporation in newborns.

### SUMMARY OF THE INVENTION

An object of the present invention is at least partially to eliminate the above-described drawbacks. In particular, the object of the present invention is to improve the handling of the thermal bag in a cost-effective and simple manner.

The thermal bag according to the present invention is used to protect a newborn against hypothermia. The thermal bag has for this at least one insertion opening for inserting the body of the newborn into an interior space of the thermal bag. This interior space is surrounded by a bag wall blocking water vapor. A thermal bag according to the present invention is characterized in that the bag wall has a sensor device for determining at least one vital function of the newborn.

Features and details that are described in connection with the thermal bag according to the present invention also apply, of course, in connection with the packaging unit according to the present invention and vice versa, so that reference is and can mutually be made to the individual aspects of the invention concerning the disclosure.

The thermal bag according to the present invention is consequently based on prior-art thermal bags and thus represents a hypothermia prevention function due to a water vapor barrier or an evaporation barrier. The interior space is provided with a limited volume, which becomes saturated relatively rapidly due to evaporation of moisture on the skin of the newborn's body. Further evaporation is stopped beginning from the time of saturation, so that cold generated by evaporation cannot lead to a further drop in the body temperature of the newborn any longer. A heat insulation is obtained in this manner for the newborn. This heat insulation is expanded by a reduction of convective cooling and of cooling by radiation.

In addition to the primary heat insulation functionality, there is a sensor functionality in the thermal bag in the manner according to the present invention. While the sensor system usually had to be attached to the body of the newborn with great effort in prior-art thermal bags, the sensor system is part of the thermal bag, namely, a component of the bag wall, in the manner according to the present invention. The bag wall may consequently have the sensor device integrally or in a combined manner. For example, the bag wall may form individual sensor surfaces, which provided electric conductivity and hence the sensor functionality due to a corresponding selection of the material, especially a plastic. The sensor device may also have sensor cables, which extend within the bag wall.

It should be noted, in principle, that an integral design of the individual sensor elements of the sensor device is, in principle, just as possible as a bonding or other manner of fastening of individual sensor elements on the bag wall.

It is important that after the body of the newborn has been inserted into the interior space, the vital function can be determined by means of the sensor device. The sensor device is configured especially for contacting the body of the newborn in at least some sections.

A possible vital function is, for example, the monitoring of the heart rate of the newborn. For example, the sensor device may have for this two sensor surfaces, which are formed in the bag wall, electrically insulated from one another and at spaced locations from one another. When the body is inserted into the interior space, the newborn is then placed on a support together with the thermal bag. An electric contact is thus automatically established between the body and the respective sensor surface of the sensor device due to the weight of the newborn's body. The corresponding electrical determination of the heart rate can now be performed by the sensor device in the known manner. The possibility of electrical contacting is supported here by the atmosphere within the interior space of the thermal bag, which atmosphere is usually saturated with moisture, and by the moist surface of the newborn's body.

However, the determination of at least one vital function is not limited, of course, to a compulsory contact with the newborn's body. Thus, the sensor device may also have, for example, other sensor functionalities, especially for the contactless determination of at least one vital function. It is, of course, also possible to determine, in a combined manner, a plurality of different vital functions according to the present invention.

The heat insulation functionality of prior-art thermal bags may consequently be combined according to the present invention with an improved vital function monitoring. In particular, important vital functions can be monitored essentially without interruption, because monitoring is made possible by the sensor device even during the transportation of the newborn within the thermal bag from a delivery room to an intensive care unit. In addition, automatic contact and hence an automatic arrangement of the sensor device in the orientation needed for the mode of operation can preferably be brought about. This leads to a further improvement, especially in hectic birth situations. The vital function monitoring is thus ensured without risks or with a lower risk.

It may be advantageous if the sensor device in a thermal bag according to the present invention has at least one interface for establishing a signal-communicating connection. This interface may be a mechanical interface and/or an electrical interface. For example, the main transmission functionality or the main analysis functionality is provided in a corresponding transmission unit and in a corresponding analysis unit, respectively. A corresponding signal-communicating connection may be made available via an electrical interface. However, a separate wireless transmission unit may also be provided, which is also fixed mechanically at the interface in addition to a signal-communicating connection. For example, such a mechanical interface may thus be configured as a Velcro fastener, as a positive-locking section or as a pushbutton connection. The data determined for the particular vital function can thus subsequently be transmitted by the transmission unit to an analysis unit in a wired manner and/or by means of a wireless module. For example, a central analysis unit, which receives the vital function data from the sensor device and the transmission device connected thereto in a wireless manner, may be provided in the delivery room. The interface may be arranged either directly at the respective sensor device or the respective sensor surface and indirectly and in a wired manner at a spaced location from the sensor surface. Furthermore, it is possible that a parameter of the vital function is displayed in the vicinity of the thermal bag on a corresponding device. A display device as a part of the thermal bag is also conceivable within the framework of the present invention.

It is advantageous, furthermore, if the sensor device in a thermal bag according to the present invention has at least one sensor surface on the inside of the bag wall, especially if the inside of the bag wall is configured in some sections as a sensor surface. The inside correspondingly makes it possible to ensure or make possible an automatic or basically possible contacting between the sensor surface and the skin of the newborn's body. The bag wall may be formed, for example, by an injection molding method. In particular, a material combination in which electrically conductive plastics form the respective sensor surface in the injection molding process can be made available especially by two-component injection molding. This leads to an especially cost-effective and simple manufacturing possibility. The sensor surface will also fail to limit the flexibility of the bag wall in this manner, so that the possibility of use when inserting the newborn's body is also limited to a very low extent only or it remains fully unhindered in this way.

It is likewise advantageous if, in a thermal bag according to the present invention, the interior space has a longitudinal axis along which the newborn's body can be received. The sensor device has at least two sensor surfaces here, which are located at spaced locations from one another axially along this longitudinal axis. This leads to the possibility of determining, for example, the heart rate. The spaced locations of the two sensor surfaces from one another allows for an electrical insulation of these two sensor surfaces, which is necessary for determining the heart rate. At the same time, the individual sensor surfaces may be configured as especially large surfaces, so that the probability of the corresponding contacting of the newborn's body is increased. The longitudinal axis within the interior space preferably also pertains to a corresponding longitudinal axis of the newborn's body, which axis extends along the torso essentially along the newborn's spine. The body may preferably be arranged in such a way that the sensor surfaces enclose the newborn's heart in an ideal orientation in order to make it possible to make available especially accurate data for determining the heart rate as a vital function. The two sensor surfaces are preferably arranged at an angle in relation to one another, which angle is oriented along a pitch angle of the heart in relation to the longitudinal axis described. A heart is usually oriented from top left to bottom right in the top view of the infant's face and forms in this way the corresponding pitch angle of the heart. The measurable amplitude is improved in this way even further for the heart rate as a vital function.

It is likewise advantageous if the sensor device, especially at least one sensor surface, is arranged in a thermal bag according to the present invention on the rear side of the inside of the bag wall, which rear side faces the newborn's back. In other words, the thermal bag is orientation-specific. It has a top side and a rear side, and with the newborn's body inserted, the newborn lies with his back on the rear side of the inner wall. It is thus ensured by the orientation specificity that the infant's back makes available a contacting of the individual sensor surfaces, which contacting was already described several times, due to the body's own weight. The automatic contact and the automatic monitoring of the vital function thus quasi becomes possible. For example, a graphic display in the form of a mark may, of course, show the correct orientation for this desired orientation.

It may, furthermore, be advantageous if the bag wall in the thermal bag according to the present invention has at least one mark, which indicates the orientation of the bag wall in relation to the newborn's body with reference to the orientation of the bag wall that is necessary for the function of the sensor device. It may be, for example, the correct orientation between the front side and the rear side of the thermal bag. The insertion opening can be explicitly distinguished from other openings by the mark even if a plurality of openings are provided within the thermal bag. The mark can also be useful in this way in case of a corresponding orientation in relation to the longitudinal axis, especially if a plurality of sensor surfaces are oriented to the corresponding pitch angle axis of the heart in relation to the longitudinal axis. As a result, the thermal bag can be used as optimally as possible even if the staff is trained less thoroughly and in hectic birth situations. Partial outlines or outlines of the infant on the top side of the thermal bag are also possible as marks in addition to color codes or color marks.

It is advantageous, moreover, if at least one manipulation opening, which is arranged especially opposite the insertion opening, is provided in a thermal bag according to the present invention. In other words, the thermal bag may be configured in such an embodiment as a so-called tube-like bag. It has two openings located opposite each other, and the manipulation opening makes possible the primary medical care from the side opposite the infant's head. For example, additional sensors can thus be arranged through the manipulation opening. Other medical interventions, for example, catheterization or similar procedures, can also be performed through the manipulation opening. It is thus also possible to facilitate the manufacture of the thermal bag in a continuously proceeding manner. The tube-like bag can thus be manufactured quasi from the roll and subsequently cut and packaged in individual steps. This leads to a marked reduction of the manufacturing costs and hence in the price of the thermal bag according to the present invention.

It is likewise advantageous if the bag wall in the thermal bag according to the present invention is formed at least in some sections and especially entirely from a plastic material. This likewise leads to a reduction of the manufacturing cost. For example, the thermal bag may thus be configured as a disposable thermal bag. Manufacture may be possible, for example, for blow films according to extrusion methods. It should be noted in this connection that the sensor device may also be able to be manufactured, at least partly, in the form of electrically conductive plastics according to the extrusion method, quasi as a two-component injection molding operation.

Another advantage can be gained if the sensor device in a thermal bag according to the present invention is configured for the determination of at least one of the following vital functions of the newborn:
Heart rate
Trend of heart rate
Oxygen saturation
Body temperature.

The above list is not a comprehensive one. A determination of a combination of a two or more of the above-mentioned vital functions or other vital functions may, of course, also be provided by the sensor device. The combined monitoring of different vital functions by common sensor surfaces is also possible in the sense of the present invention. The trend of the oxygen saturation and the trend of the body temperature may, of course, also be detected as an independent vital function.

The present invention also pertains to a packaging unit, having a sterile outer packaging as well as a thermal bag according to the present invention arranged within the sterile outer packaging. The packaging unit according to the present invention thus offers the same advantage as those explained in detail in reference to a thermal bag according to the present invention.

Further advantages, features and details of the present invention appear from the following description, in which exemplary embodiments of the present invention are described n detail with reference to the drawings. The features mentioned in the claims and in the specification may be essential for the present invention both in themselves or in any combination.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a first embodiment of a thermal bag according to the present invention;

FIG. 2 is a schematic view showing the embodiment according to FIG. 1 after insertion of the newborn;

FIG. 3 is a schematic view showing another embodiment of a thermal bag according to the present invention;

FIG. 4 is a schematic sectional view showing another embodiment of a thermal bag according to the present invention;

FIG. 5 is a schematic sectional view showing another embodiment of a thermal bag according to the present invention; and

FIG. 6 is a schematic sectional view showing an embodiment of a packaging unit according to the present invention.

DESCRIPTION OF THE PREFERRED EMBODIMENTS

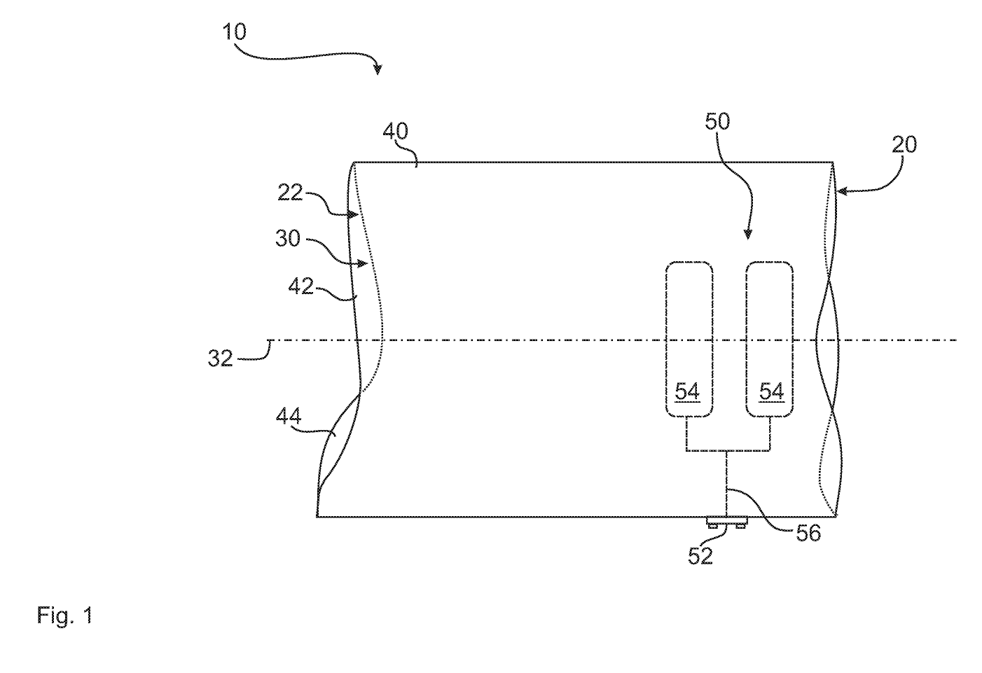

FIG. 1 shows a first embodiment of a thermal bag 10 according to the present invention. This bag is configured as a tube-like bag, which has an insertion opening 20 on the right-hand side and a manipulation opening 22 on the left-hand side. An interior space 30 is formed by a circumferential bag wall 40, for which a material acting as a water vapor barrier is selected. This interior space 30 is provided with an inner wall. The top view according to FIG. 1 shows partly directly the inside 42 of the bag wall 40 and partly the rear side 44 of said inside 42.

The two openings 20 and 22 are located opposite each other in reference to the longitudinal axis 32 of the thermal bag 10.

The thermal bag 10 according to FIG. 1 is equipped with a sensor device 50. This sensor device 50 forms two sensor surfaces 54, which are arranged axially along the longitudinal axis 32 at spaced locations from one another. A signal-communicating connection is made available via a signal cable 56 to an interface 52.

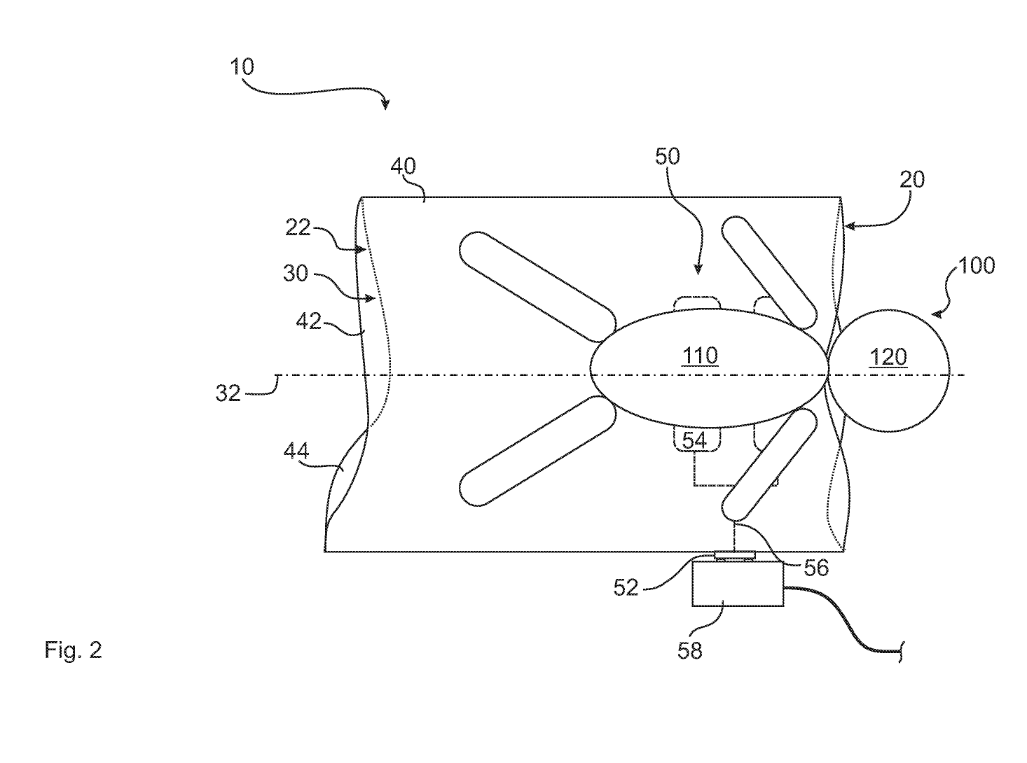

A thermal bag 10 according to FIG. 1 is used following the birth of a newborn, for example, a premature infant. The newborn 100 is inserted with his or her body 110 into the interior space 30 of the thermal bag, as this is shown in FIG. 2. The head 120 of the newborn 110 remains outside the interior space 30. It can also be clearly seen that the back of the body 110 of the newborn 100 comes to lie on the sensor surfaces 54. At the same time or before, a signal-communicating contacting of the interface 52 is established with a transmission unit 58, which transmits the determined vital function data, in a wired manner in this case, to an analysis unit, not shown. The interface 52 is configured in this case in a mechanical and electrically signal-communicating manner, so that, for example, two pushbuttons make it possible to mechanically fix the transmission unit 58.

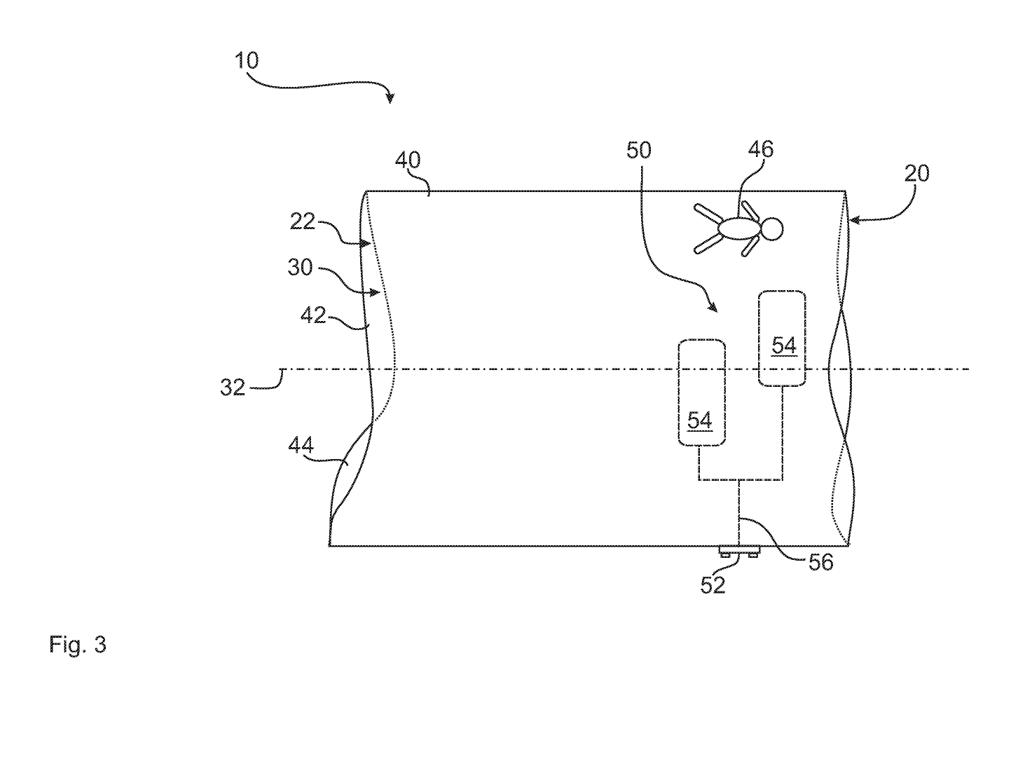

FIG. 3 shows a variant of the embodiment of the thermal bag 10 according to FIG. 1. The two sensor surfaces 54 are offset here in the circumferential direction of the bag wall 40. This offset depends on a corresponding pitch angle of the heart of the newborn 100 in relation to the longitudinal axis 32. The amplitude of the determined data of the vital function related to the heart rate of the newborn 100 can thus be improved.

Further, it can be seen in the embodiment according to FIG. 3 that a mark 46 is arranged on the top side of the bag wall 40. Due to its positioning, it indicates the top side, so that the sensor surfaces 54 can, indeed, provide for the desired contacting with the back of the infant, thus generated by the own weight of the newborn 100. In addition, the correct orientation of the head side and hence the definition of the insertion opening 20 is given by the mark 46 in a simple and above all unambiguous manner.

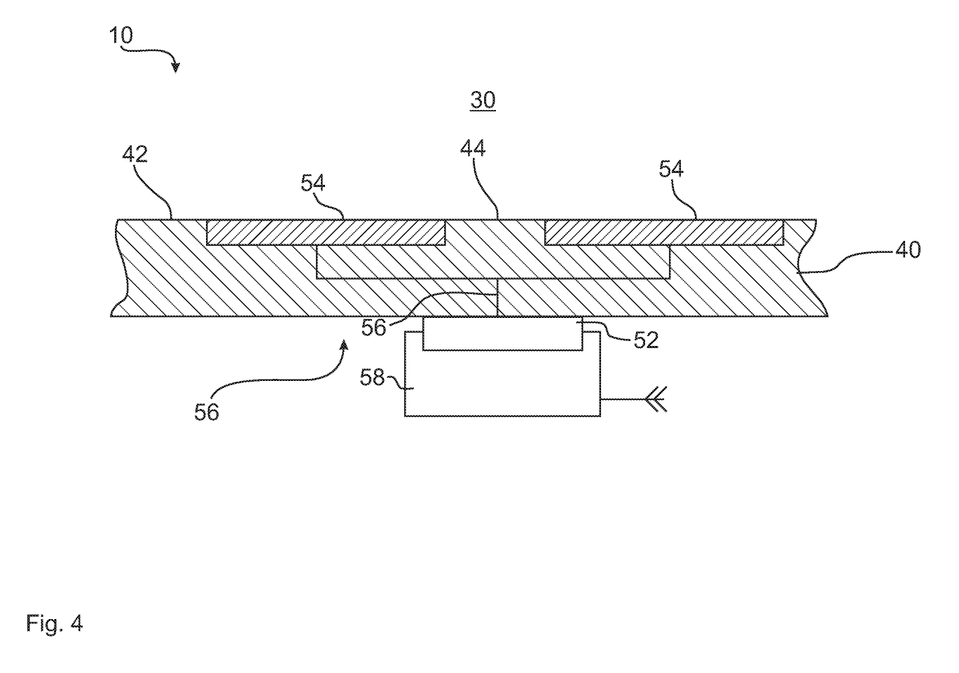

FIG. 4 schematically shows the cross section of a bag wall 40. It can be clearly seen here that if, for example, plastic material is used according to the extrusion method, the sensor surfaces 54 may likewise be manufactured from electrically conductive plastic material together with the bag wall 40. The individual signal cables 56 may extend within the material of the bag wall 40 or bonded to same. The interface 52 is configured in this case as a positive-locking section, so that a corresponding transmission unit 58, provided with an antenna here, can be placed.

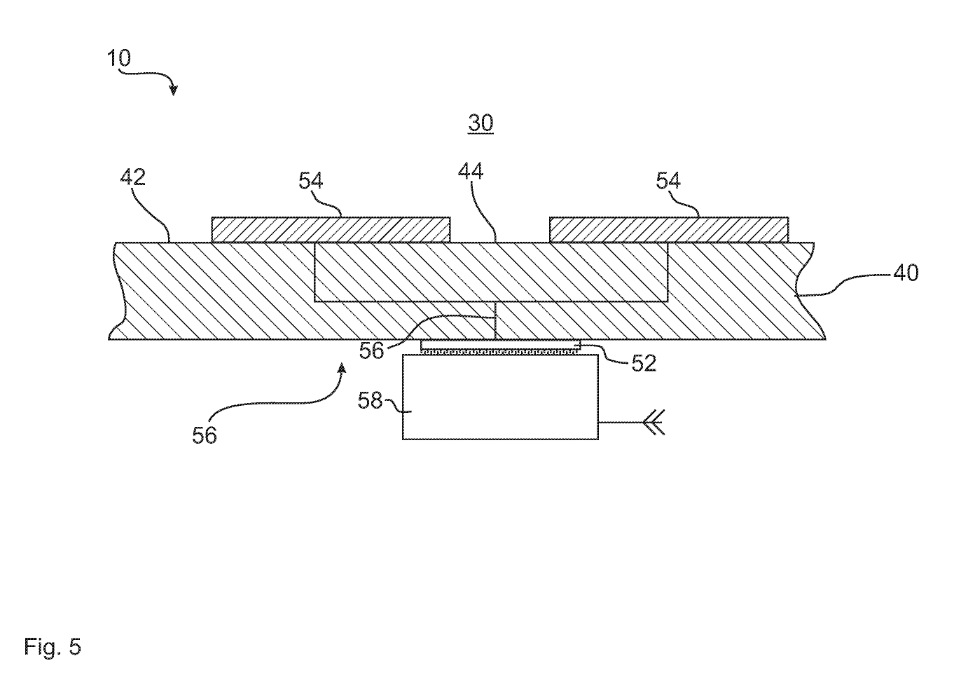

FIG. 5 shows another embodiment of a thermal bag 10 according to the present invention. The two sensor surfaces 54 are bonded here on the rear side 44 and hence also on the inside 42 in the interior space 30. Moreover, the interface 52 for the mechanical fixation is configured in this case as a Velcro fastener.

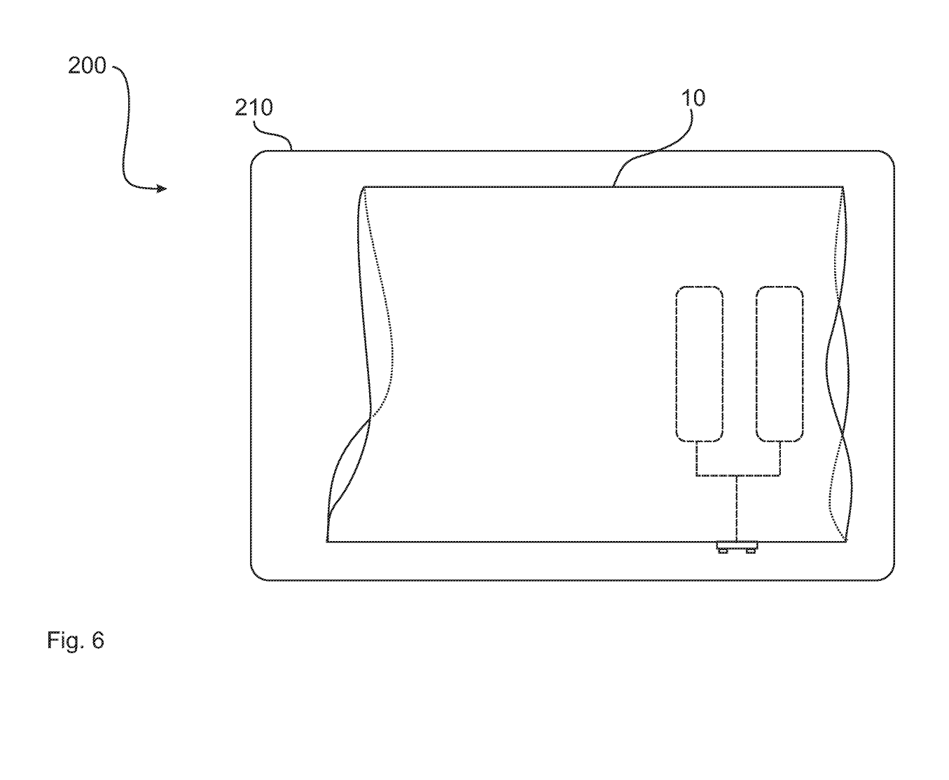

FIG. 6 schematically shows an embodiment of a packaging unit 200 according to the present invention. A thermal bag 10 according the embodiment shown in FIG. 1, arranged in a sterile manner, is located in an outer packaging 210. The outer packaging 210 is torn open for use, so that access to the thermal bag according to the present invention and the corresponding use become possible.

The above explanation of the embodiment describes the present invention exclusively within the framework of examples. Individual features of the embodiment, if technically meaningful, may, of course, be freely combined with one another without going beyond the scope of the present invention.

While specific embodiments of the invention have been shown and described in detail to illustrate the application of the principles of the invention, it will be understood that the invention may be embodied otherwise without departing from such principles.

1. A thermal bag for protecting a newborn against hypothermia, the thermal bag comprising:
a water vapor blocking bag wall defining an interior space; and
at least one insertion opening for inserting the body of the newborn into the interior space of the thermal bag, wherein the interior space is at least partially surrounded by the water vapor blocking bag wall, wherein the bag wall further comprises a sensor device configured to determine at least one vital function of the newborn.

2. A thermal bag in accordance with claim 1, wherein the sensor device has at least one interface for establishing a signal-communicating connection.

3. A thermal bag in accordance with claim 1, wherein the sensor device has at least one sensor surface on the an inside of the bag wall, the inside of the bag wall being configured as a sensor surface in some sections.

4. A thermal bag in accordance with claim 1, wherein the interior space has a longitudinal axis, along which the body of the newborn is received, wherein the sensor device has at least two sensor surfaces, which are located at spaced locations from one another axially along this the longitudinal axis.

5. A thermal bag in accordance with claim 1, wherein the sensor device comprises at least one sensor surface of the sensor device and is arranged on the a rear side of the inside of the bag wall, which said rear side faces the back of the newborn.

6. A thermal bag in accordance with claim 1, wherein the bag wall has further comprises at least one mark, which indicates the orientation of the bag wall in relation to the body of the newborn with reference to the orientation of the bag wall that is necessary for the function of the sensor device.

7. A thermal bag in accordance with claim 1, further comprising at least one manipulation opening arranged opposite the insertion opening.

8. A thermal bag in accordance with claim 1 wherein the bag wall is made, at least in some sections, of a plastic material.

9. A thermal bag in accordance with claim, wherein the sensor device is configured to determine at least one of the following vital functions of the newborn:
heart rate;
trend of heart rate;
oxygen saturation; and
body temperature.

10. A packaged unit haviing comprising:
sterile outer packaging;
a thermal bag arranged within the outer packaging, the thermal bag comprising:
a water vapor blocking bag wall defining an interior space; and
at least one insertion opening for inserting a body of a newborn into the interior space of the thermal bag, wherein the interior space is at least partially surrounded by the water vapor blocking bag wall, wherein the bag wall further comprises a sensor device determining at least one vital function of the newborn.

11. A packaged unit in accordance with claim 10, wherein the sensor device comprises a signal interface configured to establish a signal-communicating connection.

12. A packaged unit in accordance with claim 10, wherein the sensor device comprises a sensor surface and an inside of the bag wall comprises a section configured as the sensor surface.

13. A packaged unit in accordance with claim 10, wherein the interior space has a longitudinal axis, along which the body of the newborn is received, wherein the sensor device has at least two sensor surfaces located at spaced locations from one another axially along the longitudinal axis.

14. A packaged unit in accordance with claim 10, wherein the sensor device comprises at least one sensor surface arranged on a rear side of an inside of the bag wall, which said rear side faces the back of the newborn.

15. A packaged unit in accordance with claim 10, wherein the bag wall further comprises at least one mark, which indicates a bag wall orientation in relation to the body of the newborn with reference to the orientation of the bag wall that is necessary for a function of the sensor device.

16. A packaged unit in accordance with claim 10, further comprising at least one manipulation opening arranged opposite the insertion opening.

17. A packaged unit in accordance with claim 10, wherein the bag wall comprises sections formed of a plastic material.

18. A packaged unit in accordance with claim 10, wherein the sensor device is configured to determine at least one of
heart rate;
trend of heart rate;
oxygen saturation; and
body temperature as vital functions of the body of the newborn inserted into the thermal bag.

19. A neonate thermoregulation bag for protecting a body of a newborn against hypothermia, the thermoregulation bag comprising:
a bag wall forming an interior space and forming, at least in some sections, a water vapor barrier between the interior space and an environment of the thermoregulation bag, the bag wall further defining at least one insertion opening for inserting the body of the newborn into the interior space of the thermal bag; and
a sensor device configured to determine at least one vital function of the newborn, the sensor being integrated in a portion of the bag wall.

20. A neonate thermoregulation bag in accordance with claim 19, wherein:
the sensor device comprises a signal interface configured to establish a signal-communicating connection;
the sensor device comprises a sensor surface and an inside of the bag wall comprises a section configured as the sensor surface;
the bag wall comprises sections formed of a plastic material; and
the sensor device is configured to determine at least one of:
heart rate;
trend of heart rate;
oxygen saturation; and
body temperature as vital functions of the body of the newborn inserted into the thermal bag.
